(19) United States
(12) Patent Application Publication
(10) Pub. No.: US 2015/0104028 A1
(43) Pub. Date: Apr. 16, 2015

(54) MEASURING DEVICE

(71) Applicant: Euclid Technology Co., Ltd., Zhongli City (TW)

(72) Inventors: Ching-Sheng TWU, Zhongli City (TW); Fu-Kai CHUANG, New Taipei City (TW); Fang-Yu TU, Pingzhen City (TW); Hsiao-Ying PENG, Zhongli City (TW)

(73) Assignee: Euclid Technology Co., Ltd., Zhongli City (TW)

(21) Appl. No.: 14/139,183

(22) Filed: Dec. 23, 2013

(30) Foreign Application Priority Data
Oct. 11, 2013 (TW) 102136866

(51) Int. Cl. H04R 1/46; A61B 7/04; H03G 3/00

(52) U.S. Cl. CPC H04R 1/46; H03G 3/00; A61B 7/04; H04R 2499/11
USPC 381/67

(57) ABSTRACT

A measuring device includes an audio collection module, an audio player module and a switch module. The audio player module electrically connects to the audio collection module, and the switch module electrically connects to the audio player module. The audio collection module collects sounds to generate an audio signal. The audio player module plays the audio signal. The switch module sets an enabling signal for controlling the audio player module. The enabling signal is set to be at either a first state or a second state different from the first state. When the enabling signal is at the first state, the audio player module plays the audio signal under a first output mode. When the enabling signal is at the second state, the audio player module plays the audio signal under a second output mode different from the first output mode.

### CROSS-REFERENCE TO RELATED APPLICATIONS

This non-provisional application claims priority under 35 U.S.C. §119(a) on Patent Application No(s). 102136866 filed in Taiwan, R.O.C. on Oct. 11, 2013, the entire contents of which are hereby incorporated by reference.

### TECHNICAL FIELD

The disclosure relates to a measuring device and more particularly to a measuring device determining its output according to its input.

### BACKGROUND

Most household measuring devices on the market measure only one kind of data and can not measure and record complex physiological phenomena and even provide relative suggestions to users in real time. For example, many products for measuring blood pressure or pulsation on the market only determine whether the user's blood pressure or pulsation is too high or low. However, it can not only use the measuring result of the blood pressure or the pulsation to diagnose other cardiovascular diseases and respiratory diseases. Thus, doctors always use the measurement result of a stethoscope to diagnose such diseases.

Generally, it must need skilled operators to properly operate such a stethoscope and determine the relation between the recorded sounds and diseases by using the measurement result of the stethoscope. Therefore, it may be difficult for an unskilled operator to operate a stethoscope to measure his or her own health states properly, and can even damage his or her eardrums if it is operated improperly

### SUMMARY

According to an embodiment, a measuring device includes an audio collection module, an audio player module and a switch module. The audio player module electrically connects to the audio collection module, and the switch module electrically connects to the audio player module. The audio collection module collects sounds to generate an audio signal. The audio player module plays the audio signal. The switch module sets an enabling signal for controlling the audio player module. The enabling signal is set to be at either a first state or a second state different from the first state. When the enabling signal is at the first state, the audio player module plays the audio signal under a first output mode. When the enabling signal is at the second state, the audio player module plays the audio signal under a second output mode different from the first output mode.

### BRIEF DESCRIPTION OF THE DRAWINGS

The present invention will become more fully understood from the detailed description given hereinbelow along with the accompanying drawings which are for illustration only, thus are not limitative of the present invention, and wherein:

FIG. 1A is a side view of a measuring device according to an embodiment of the disclosure;

FIG. 1B is a block diagram of the measuring device according to an embodiment of the disclosure;

FIG. 2A is a block diagram of the audio collection module in FIG. 1B according to an embodiment of the disclosure;

FIG. 2B is a block diagram of the audio collection module in FIG. 1B according to an embodiment of the disclosure;

FIG. 2C is a block diagram of the audio collection module in FIG. 1B according to an embodiment of the disclosure;

FIG. 3A is a side view of the measuring device according to an embodiment of the disclosure; and

FIG. 3B is a side view of the measuring device according to an embodiment of the disclosure.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawings.

The disclosure provides a measuring device which can collect and record heart sounds or breathing sounds when the measuring device is pressed, and thus the measuring device can be applicative to an electric stethoscope. The operation of such an electric stethoscope can refer to general medical staffs' operational habit, whereby unskilled or skilled operators can operate this electric stethoscope. Moreover, this electric stethoscope can be set to operate under a specific operation mode for playing audio signals under, or to operate under another specific operation mode for changing the volume of audio signals, whereby the disclosure can protect operators from causing damage to their eardrums.

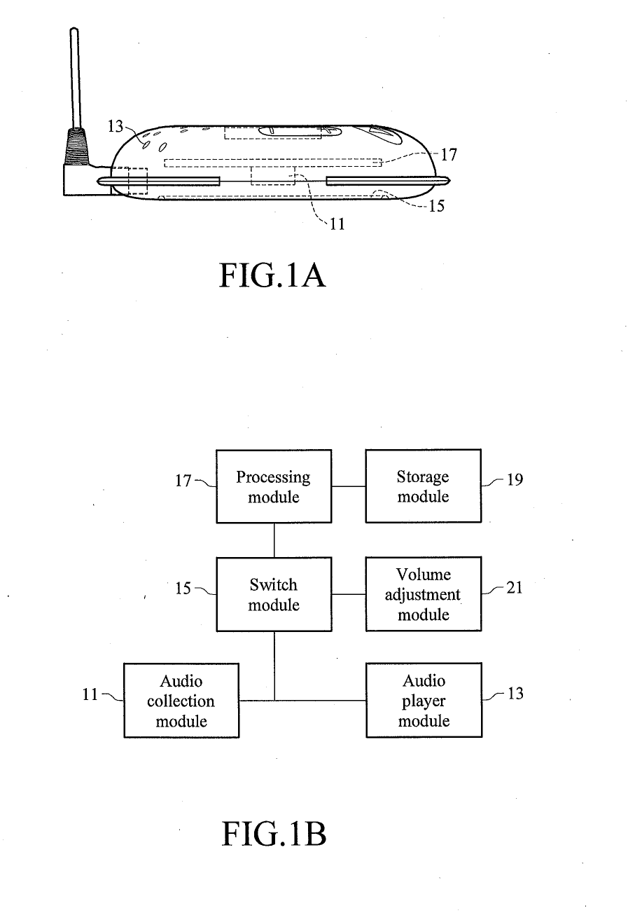

As shown in FIG. 1A, the measuring device includes an audio collection module 11, an audio player module 13 and a switch module 15. The audio player module 13 electrically connects to the audio collection module 11 and the audio player module 13 as shown in FIG. 1B.

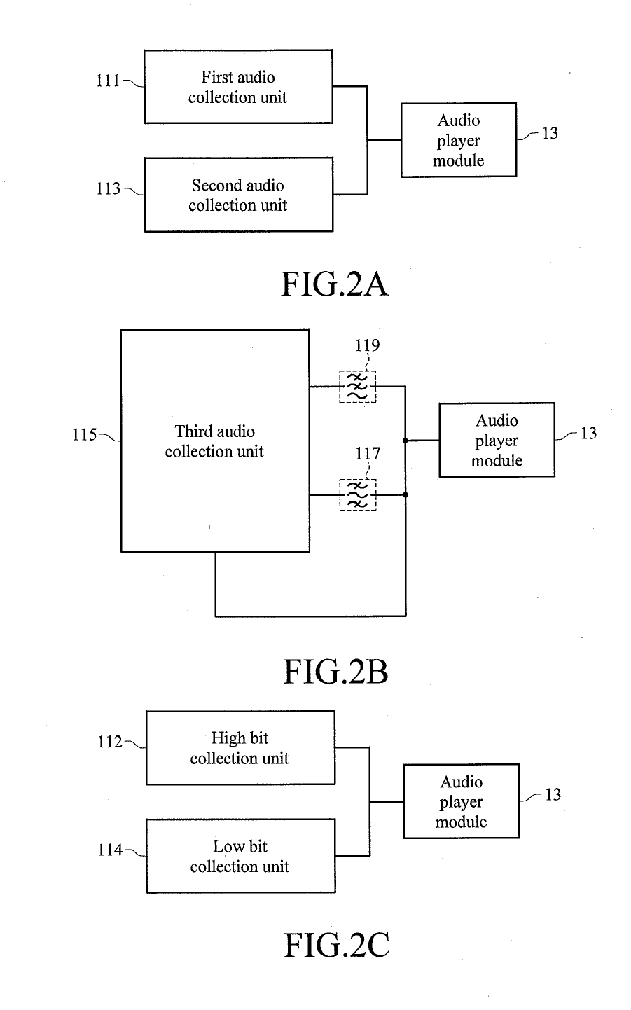

The audio collection module 11 can collect sounds to generate audio signals. In particular embodiments, the audio signal includes a first sub-band signal, e.g. an audio signal having a frequency range between 200 and 2000 Hz, and a second sub-band signal, e.g. an audio signal having a frequency range between 20 and 1000 Hz. Specifically, refer to FIG. 2A, the audio collection module 11 includes a first audio collection unit 111 and a second audio collection unit 113 both of which electrically connect to the processing module 15 wiredly or wirelessly. The first audio collection unit 111 can collect sounds to generate a first sub-band signal, and the second audio collection unit 113 can collect sounds to generate a second sub-band signal. As an example and not by way of limitation, the first audio collection unit 111 collects sounds at a high frequency range between 200 and 2000 Hz, and the second audio collection unit 113 collects sounds at a low frequency range between 20 and 1000 Hz. As an example and not by way of limitation, the first audio collection unit 111 can be a diaphragm chest piece, an audio collector having a band-pass filter, or any suitable audio collector capable of collecting high frequency range sounds. As an example and not by way of limitation, the second audio collection unit 113 can be a bell type chest piece, an audio collector having a low-pass filter, or any suitable audio collector capable of collecting low frequency range sounds.

In particular embodiments, refer to FIG. 2B, the audio collection module 11 includes a third audio collection unit 115, a first filter unit 117 (e.g. a band-pass filter) and a second filter unit 119 (e.g. a low-pass filter). The third audio collection unit 115 electrically connects to the processing module 15 directly or through the first filter unit 117 and/or the second filter unit 119. The third audio collection unit 115 can directly collect sounds (which human ears can listen to) to generate an audio signal, and the audio signal is filtered by the first filter unit 117 to become a first sub-band signal with a frequency range, for example, between 200 and 2000 Hz, or is filtered by the second filter unit 119 to become a second sub-band signal with a frequency range, for example, between 20 and 1000 Hz. After receiving audio signals, the processing module 15 can process and transmit a part or all of them. The audio collection module 11 can be designed according to particular requirements. As an example and not by way of limitation, the third audio collection unit 115 can be a dynamic microphone, a condenser microphone, an electret condenser microphone, a micro-electrical-mechanical system (MEMS) microphone, a ribbon microphone, a carbon microphone, a piezoelectric microphone, a fiber optic microphone, a laser microphone, a liquid microphone or any suitable analog-to-digital converter (ADC) for collecting sounds. As an example and not by way of limitation, the first filter unit 117 and the second filter unit 119 can be analog or digital filters.

The audio player module 13 can plays the audio signals. In particular embodiments, the audio player module 13 can include one or more digital-to-analog converters (DACs), and a number of bits accessed by the audio player module 13 is equal to a number of bits of the digital audio signal outputted by the audio collection module 11. As an example and not by way of limitation, when the audio signal outputted by the audio collection module 11 has 8 bits, the audio player module 13 will be an 8-bit DAC. As an example and not by way of limitation, the audio player module 13 can be a serial or parallel DAC, and the input of the audio player module 13 can correspond to the output of the audio collection module 11. Moreover, the audio player module 13 can further include one or more audio output ports (e.g. 3.5 millimeter earphone jacks) for connecting to external audio players.

The switch module 15 can set an enabling signal for controlling the audio player module 13. As an example and not by way of limitation, the enabling signal is at either a first state or a second state different from the first state. When the enabling signal is at the first state, the audio player module 13 plays the audio signal under a first output mode, and when the enabling signal is at the second state, the audio player module 13 plays the audio signal under a second output mode different from the first output mode. As an example and not by way of limitation, the first output mode indicates that the audio signal will be played at a relatively lower volume, and the second output mode indicates that the audio signal will be played at relatively higher volume. As another example and not by way of limitation, the first output mode indicates that the audio signal will be played with a fixed volume, and the second output mode indicates that the volume of the played audio signal can be changed.

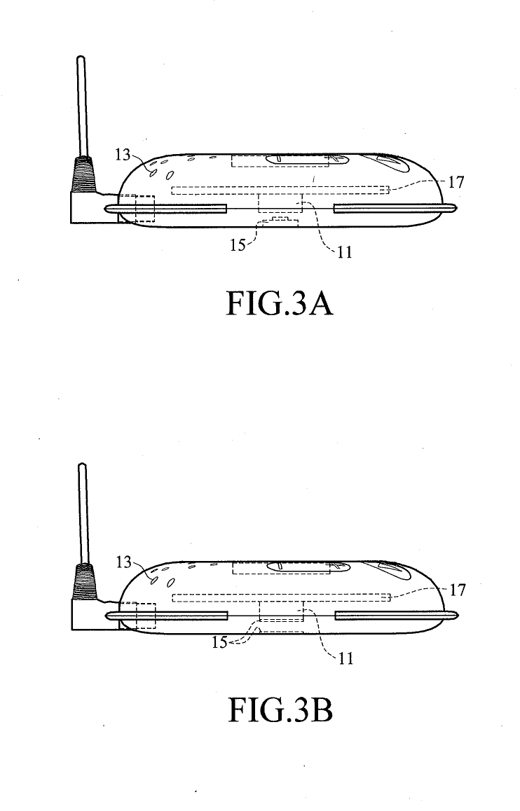

Refer to FIG. 1A, the switch module 15 and the audio collection module 11 are at a measuring terminal of the measuring device, i.e. close to the bottom surface of the measuring device in the drawing. In a particular embodiment, refer to FIG. 3A, the switch module 15 can be a button under the audio collection module 11, and the bottom surface of the measuring device is flexible. If the measuring device is pressed at a proper location, the switch module 15 will contact with the audio collection module 11, which is equivalent to a pressed button. In contrast, if the measuring device has not been pressed, the equivalent bottom will not be pressed. Thus, the switch module 15 sets the enabling signal to be at the first state when the bottom has not been pressed, and the switch module 15 sets the enabling signal to be at the second state when the bottom has been pressed.

As another example and not by way of limitation, the switch module 15 in FIG. 3B is embodied by two opposite metallic films respectively disposed under the audio collection module 11 and inside the inner surface of the bottom of the measuring device, and the bottom surface of the measuring device is flexible. When the measuring device has not been pressed, the capacitance value between the metallic films will be constant. When the measuring device is pressed at a proper location, the two metallic films will be close to each other, where a capacitance value between the metallic films will increase. Alternately, the metallic films will contact with each other, where the capacitance value become zero. Thus, the measuring device can know whether itself is pressed or not, by measuring the capacitance value between the metallic films of the switch module 15. For example, if the capacitance value between two metallic films is larger than a preset threshold or equal to zero, the switch module 15 will know that the measuring device has been pressed, where the enabling signal will be set to be at the second state. If the capacitance is less than the preset threshold and is not equal to zero, the switch module 15 will know that the measuring device has not been pressed, where the enabling signal will be set to be at the first state.

In an embodiment, refer to FIG. 1B, the measuring device can further include a processing module 17 electrically connecting to the switch module 15. When the enabling signal is at the first state, the processing module 17 is disabled, and when the enabling signal is at the second state, the processing module 17 is enabled to, according to a built-in algorithm, process the audio signal outputted by the audio collection module 11. As an example and not by way of limitation, the algorithm can include debugging, digital filtering or any suitable algorithm for processing audio signals. As an example and not by way of limitation, the algorithm can be provided by an application portability profile (APP). As an example and not by way of limitation, the processing module 17 can be a central processing unit (CPU), a single-chip processor or any suitable electric device having computing, logic processing and controlling abilities.

Moreover, the measuring device can further include a storage module 19 electrically connecting to the processing module 17. When the enabling signal is at the second state, the processing module 17 stores the processed audio signal in the storage module 19. Thus, the stored audio signals can be read out then. In some embodiments, the audio player module 13 can connect to the processing module 17 or the storage module 19. As an example and not by way of limitation, the first output mode indicates that the audio signal will be played at a constant volume, and the second output mode indicates that the audio signal will not be played but stored in the storage module 19 directly or through the processing module 17.

As an example and not by way of limitation, the audio collection module 11 can include an ADC whose output has (M+N) bits, where M and N are integers larger than 1. As an example and not by way of limitation, the ADC includes a high bit collection unit 112 processing M high-level bits, and a low bit collection unit 114 processing N low-level bits, where the high-level bits indicate the bits closest to the most significant bit (MSB) in a (M+N)-bit binary number and the low-level bits indicate the bits closest to the least significant bit (LSB) in the binary number. If the enabling signal is at the first state, only the high bit collection unit 112 is enabled, where the M high-level bits of the (M+N) bits will be outputted and the N low-level bits of the (M+N) bits will be ignored or blocked. Herein,

N high-level bits of the (M+N) bits inputting into the audio player module 13 will become low, and M low-level bits in the (M+N) bits will become high as the same as the M high-level bits outputted by the audio collection module 11. Thus, the operator can listen to the sound of the audio signal at the current volume. On the other hand, if the enabling signal is at the second state, both of the high bit collection unit 112 and the low bit collection unit 114 are enabled, where all the (M+N) bits will be outputted from the audio collection module 11. Herein, the (M+N) bits inputting into the audio player module 13 respectively correspond to the (M+N) bits outputted by the audio collection module 11. Thus, the operator can listen to more details of the audio signal.

In an embodiment, the measuring device can further include a volume adjustment module 21 which electrically connects to the audio player module 13 and can change the volume of audio signals according to a modulation signal. If the enabling signal is at the first state, the volume adjustment module 21 will be disabled, where the output volume can not be changed via the volume adjustment module 21. If the enabling signal is at the second state, the volume adjustment module 21 will be enabled, where the output volume can be changed via the volume adjustment module 21. Therefore, it can protect operators from causing damage to their eardrums by setting the volume too high or collecting sounds with too high volume when the enabling signal is at the first state (i.e. the measuring device has not been pressed).

As set forth above, the disclosure can either allow operators to increase the volume of the audio signal played, or play the audio signal at a fixed relative lower volume, so as to protect the operators from causing damage to their eardrums.

What is claimed is:

1. A measuring device, comprising:
an audio collection module, configured to collect sounds to generate an audio signal;
an audio player module, electrically connecting to the audio collection module and being configured to play the audio signal; and
a switch module, electrically connecting to the audio player module and being configured to set an enabling signal for controlling the audio player module;
wherein the enabling signal is at either a first state or a second state different from the first state, the audio player module plays the audio signal under a first output mode when the enabling signal is set to be at the first state, and the audio player module plays the audio signal under a second output mode different from the first output mode when the enabling signal is set to be at the second state.

2. The measuring device according to claim 1, further comprising a processing module, electrically connecting to the switch module, wherein, when the enabling signal is at the first state, the processing module is disabled, and when the enabling signal is at the second state, the processing module is enabled to process the audio signal by using an algorithm.

3. The measuring device according to claim 2, further comprising a storage module, electrically connecting to the processing module, wherein, when the enabling signal is at the second state, the processing module further stores the processed audio signal in the storage module.

4. The measuring device according to claim 2, wherein the audio signal comprises a first sub-band signal and a second sub-band signal, and the audio collection module comprises:
a first audio collection unit, electrically connecting to the processing module and being configured to collect sounds at a first frequency range to generate the first sub-band signal; and
a second audio collection unit, electrically connecting to the processing module and being configured to collect sounds at a second frequency range different from the first frequency range to generate the second sub-band signal.

5. The measuring device according to claim 2, wherein the audio signal comprises a first sub-band signal and a second sub-band signal, and the audio collection module comprises:
a third audio collection unit, configured to collect sounds to generate the audio signal;
a first filter unit, electrically connecting to the third audio collection unit and the processing module and being configured to filter the audio signal to generate the first sub-band signal; and
a second filter unit, electrically connecting to the third audio collection unit and the processing module and being configured to filter the audio signal to generate the second sub-band signal;
wherein a frequency range of the first sub-band signal is different from a frequency range of the second sub-band signal.

6. The measuring device according to claim 1, wherein the switch module sets the enabling signal when being pressed, when a pressure of pressing the switch module is less than a preset threshold, the switch module sets the enabling signal to be at the first state, and when the pressure of pressing the switch module is larger than the preset threshold, the switch module sets the enabling signal to be at the second state.

7. The measuring device according to claim 1, wherein the measuring device has a measuring terminal at which the audio collection module and the switch module are.

8. The measuring device according to claim 1, wherein the audio collection module comprises:
a high bit collection unit, electrically connecting to the switch module; and
a low bit collection unit, electrically connecting to the switch module;
wherein, when the enabling signal is at the first state, the high bit collection unit rather than the low bit collection unit is enabled to collect sounds to generate the audio signal under a first collection mode, and when the enabling signal is at the second state, the high bit collection unit and the low bit collection unit are enabled to collect sounds to generate the audio signal under a second collection mode.

9. The measuring device according to claim 1, further comprising a volume adjustment module, electrically connecting to the switch module and the audio player module and being configured to change a volume value of the audio player module according to a modulation signal when being enabled,
wherein when the enabling signal is at the first state, the volume adjustment module is disabled, and when the enabling signal is at the second state, the volume adjustment module is enabled.
